(19) United States
(12) Patent Application Publication
(10) Pub. No.: US 2003/0236541 A1
(43) Pub. Date: Dec. 25, 2003

(54) Method and system for removing antlers

(75) Inventors: Michael P. Callicrate, St. Francis, KS (US); Gordon Ross, St. Francis, KS (US); Lindsay Mathews, Hamilton (NZ)

Correspondence Address: SHERIDAN ROSS PC, 1560 BROADWAY, SUITE 1200, DENVER, CO, 80202

(73) Assignee: Michael P. Callicrate, St. Francis, KS

(21) Appl. No.: 10/176,800

(22) Filed: Jun. 21, 2002

(51) Int. Cl. A61B 1/04

(52) U.S. Cl. 606/163

(57) ABSTRACT

An apparatus and method for removing antlers from animals in a manner that is substantially painless, and thus humane, and that utilizes a particular banding tool and novel band.

### FIELD OF THE INVENTION

This invention relates in general to tools for removing body parts and in particular to a method and apparatus for removing antlers from an animal.

### BACKGROUND OF THE INVENTION

Antlers from deer, elk, or other antlered animals are put to a myriad of uses. Antlers are used in the traditional medical practices of many cultures. They are used as decorative elements in lamps, furnishings, architecture, and other products. Hunters typically use antlers to “rattle in” game during hunting season. The demand for antlers is ever-increasing due to these exemplary uses and many others. Antlers are grown each year by the adult males of all deer species found in New Zealand. They form on ‘pedicles’—permanent projections on the frontal bone of the stag's skull. The pedicles and first antler normally develop in early spring of the stag's first year, at about ten months of age. The first year antlers are normally single unbranched spikes, which is why young stags are known to farmers as ‘spikers’. Unlike horns, which are mostly made up of keratin—the protein which makes up wool, hair and hoof—antlers begin as living cartilage, well-supplied with blood, enclosed in a skin covered with fine hair. Antlers at this early stage of growth are known as velvet. Over several weeks as the antlers reach their final size, the cartilage gradually converts into bone. In the process the antler blood supply and nerves are lost. When their antlers have fully hardened, stags rub them against trees or rocks to clean off the skin which remains. They now possess a rack of sharp bony weapons ready for use in combat during the autumn rut or mating season.

Each spring the cycle begins again: The skin around the pedicle expands and grows, initiating new antler growth. The hard antler from the previous season is cast off and the growth of a new season's velvet crop begins. On a farm the deer producing the best velvet are retained. From 55 to 60 days after casting the velvet is at its most valuable stage from medicinal purposes. When velvet is harvested for use in medicines, it is removed about a centimeter above the pedicle. Like the antlers on wild stags, this remaining antler turns to bone and is eventually cast-off when the new antler grows in spring. These small remnants of antler are called ‘buttons’ by deer farmers and the time of casting is known as ‘button-drop’. Once it is removed, the velvet is allowed to cool on racks before being frozen. It is then ready to be graded and processed. If it has been removed at the correct time, before it has started to turn to bone, the entire antler may be used for medicinal purposes. Velvet removal is typically a surgical procedure undertaken by a veterinarian or a specially trained farmer that requires severe medication of the deer or other antlered animal before the procedure is performed. Deer velvet regenerates naturally every year. Velvet is removed using methods proven to ensure analgesia, usually chemical, so deer feel no pain. Non-removal of antlers presents a significant danger to deer and other stock and also handlers.

Once the chemicals necessary to anesthetize the animal are injected into the animal, it takes a few scant minutes (usually the time in which the animal reacts to the chemical) for the chemical to completely saturate the antlers of the animals. This saturation of the antler with the anesthetizing chemical is undesirable, since that chemical will then be transferred on to the end consumer. Thus, by the time the animal is fully anesthetized, the antlers are saturated and undesirable.

If humans are going to continue to consume antlers as medicinal products, what is needed is a humane, pain free, and chemical free method of removing antlers that will prevent injury to the animal and also will allow the antlers to grow back year after year.

### SUMMARY OF THE INVENTION

The present invention provides a method and an apparatus that allows for the humane, pain free and chemical free removal of antlers from an animal. The present invention can also assist in the partial removal or amputation of other body parts of an animal or human, including the surgical removal of prolapses (where an organ or a part of an organ that belongs inside the body is outside of the body) and the surgical removal of parts of or the entirety of a limb.

The present invention allows an elastomeric strap to be tightly attached to an antler (prolapse or other body part) to cut off blood supply to the antler and to deaden the nerves that are part of the antler (prolapse or other body part). Cutting off the blood supply and deadening the nerves allows the removal of the antler, prolapse or other body part, eliminating any pain felt by the animal, reducing the amount of blood loss, and reducing the likelihood of swelling, infection and/or prolonged stress to the animal. The present invention also allows the strap to be tightened quickly thereby reducing the length of time that the animal must be restrained.

According to one aspect of the present invention, a method and apparatus for removal is provided. The method includes the steps of attaching an elastomeric strap material to a removal tool and forming a loop of the elastomeric strap material around the antler to be removed. Once the loop is formed, the method includes pulling the loop using the tool to tighten the loop around the antler and clamping a device about said loop so that the loop maintains pressure about the antler until the antler can be removed through surgical means.

A separate aspect of the present invention related to a method and apparatus for using a preformed removal band attached to an endless loop in conjunction with a removal tool to remove the antler, prolapse or other body part. Use of an endless loop eliminates the need to use a linear length of banding material for forming a loop around a body part or antler.

A tool constructed in accordance with an embodiment of the present invention includes a clamping device which is removably attached to the tool and removably attached to the elastomeric strap material. The tool has a winding assembly that is connected to the strap and/or the removal band such that the loop is tightened by winding the band.

Preferably, the winding assembly includes a spool that facilitates the securing of a band thereabout as the spool is rotated. A ratchet mechanism can be employed to provide for one way rotation of the spool. In one embodiment, the tool is operated by pneumatic or electrical motors or by power tools which function to wind the band around the spool to achieve a desired loop tightness or tension. In a related embodiment, the motor or power source used to wind the tool is recessed within the handle portion of the tool and engages a worm gear or other similar mechanism to rotate the winding spool. The tightness of the loop can be modified by adjusting either the pneumatic/electrical winding device so that winding of the strap material ceases at a predetermined tension, or by providing a clutch mechanism on the tool itself. The winding assembly rotates in a direction perpendicular to the longitudinal axis of the tool effectively shortening the endless loop and applying pressure around the antler of the animal.

In another embodiment of the tool of the present invention, the tool is provided with a tensioning indicator that indicates the tension applied to an elastomeric loop during a removal procedure. An operator of this embodiment of the tool can be assured that an effective removal tension is provided to the loop. Moreover, in one embodiment of the tool, the tension indicator is provided by a compressible tool body, wherein the amount of compression indicates a corresponding tension on the loop. The compressible tool body may be provided by telescoping tool body segments, wherein one tool body segment slidably moves or compresses an insert within an outer tool body segment. Further, a compression spring may be used to provide resistance against compression forces urging the inserted body segment further into the outer tool body segment. Thus, by calibrating the extent of compression of the compression spring for various degrees of compressive force, a loop tension indicator can be provided as one or more markings on either the inner or the outer tool body segment.

In another embodiment of the tool of the present invention, the tool includes a lever which is biased against a grommet that is fitted over the elastomeric material. The pressure exerted by the lever prevent the grommet from inadvertently being mispositioned prior to and during the operation of the tool. Further more, when sufficient tension is put on the endless loop and the consequent pressure is applied to the body part to be removed, the lever is used to deform the grommet upon and/or around the endless loop to secure the loop.

One aspect of the present invention includes a removal band that facilitates the tightening of the loop about the antler or other body part. The removal band is removably attached to the spool and at least one end of the elastomeric loop. Another aspect of the present invention is a tool that includes a body, a winding mechanism attached to said body, a mount for a clamping device, a releasable locking lever to insure that the clamping device remains on the tool throughout use of the tool, and an attachment member that is adapted to be attached to the removal band or, alternatively, the elastomeric loop.

Other features and benefits of the present invention will become apparent from the detailed description with the accompanying figures contained herein.

### BRIEF DESCRIPTION OF THE DRAWINGS

The figures which follow depict a preferred embodiment of the invention, and may depict various alternative embodiments. The invention is not limited to the embodiment or embodiments depicted herein since even further various alternative embodiments will be readily apparent to those skilled in the art. For the ease of the reader, like reference numerals in various drawing figures refer to identical structural elements or components.

FIG. 1 depicts a side view of an apparatus constructed in accordance with at least one aspect of the present invention.

FIG. 2 depicts a top view of an apparatus constructed in accordance with at least one aspect of the present invention.

FIG. 3 depicts a side view of an apparatus, having a crimping device, constructed in accordance with at least one aspect of the present invention.

FIG. 4 depicts a top view of an apparatus, with a partial cutaway, having a pneumatic or hydraulic winding mechanism constructed in accordance with at least one aspect of the present invention.

FIG. 5 depicts a top view, with a partial cutaway, having a tension indicator constructed in accordance with at least one aspect of the present invention.

FIGS. 6A and 6B depict a side and top view, respectively, of a clamping device constructed in accordance with at least one aspect of the present invention.

FIGS. 7A, 7B, and 7C depict side views of the components of a clamping device constructed in accordance with at least one aspect of the present invention.

FIGS. 8A, 8B, and 8C depict top views of the components of a clamping device constructed in accordance with at least one aspect of the present invention.

FIGS. 9A, 9B and 9C depict various views of the components of another embodiment of a clamping device constructed in accordance with at least one aspect of the present invention.

FIGS. 10A, 10B, and 10C depict top views of a removal band and the attachment of an elastomeric strap material thereto, the removal band being constructed in accordance with at least one aspect of the present invention.

FIG. 11 depicts a top view of another embodiment of a removal band constructed in accordance with at least one aspect of the present invention.

FIG. 12 depicts a top view of another embodiment of a removal band constructed in accordance with at lease one aspect of the present invention.

For the ease of the reader in referring to the drawings, the following component list is provided:

| | |
|---|---|
| Tool | 10 |
| Adjustable Tool Handle | 11 |
| Winding Handle | 12 |
| Release End, Locking Lever | 13 |
| Mount End, Locking Lever | 14 |
| Clamp Device Mount | 15 |
| Winding Mechanism | 16 |
| Tool Body | 17 |
| Spool Anchor | 18 |
| Spool | 19 |
| Crimping Device | 20 |
| Grommet | 21 |
| Lever | 22 |
| Connector to Body | 23 |
| Lock | 24 |
| Elastomeric Strap Material | 30 |
| or Loop | |
| Pneumatic Device | 40 |
| Air Supply | 41 |
| Air Piston | 42 |
| Piston Rod | 43 |
| Drive Linkage | 44 |
| Tension Indicator Device | 50 |
| Tension Indicator | 51 |
| Cutout for Tension Indicator | 52 |
| Compression Spring | 53 |
| Spacer | 54 |
| Shaft | 55 |
| Retaining Member | 56 |
| Bore | 57 |
| Arrow-direction of force | 58 |
| Receptacle | 59 |
| Washer Assembly | 60 |
| Threaded Free End | 61 |
| Clamp Device | 70 |
| Pivot | 71 |
| Bar | 72 |
| Fulcrum | 73 |
| Arm | 74 |
| Anchor Screw | 75 |
| Support for Roller | 76 |
| Roller | 77 |
| Lock for Bar | 78 |
| Frame | 79 |
| Support Screws | 80 |
| Support Bar | 81 |
| Alternate Clamp Device | 90 |
| Pivot Member | 91 |
| Fulcrum | 92 |
| Locking Member | 93 |
| Support Screw | 94 |
| Support Bar | 95 |
| Frame | 96 |
| Roller | 97 |
| Anchor | 98 |
| Receiving Member | 99 |
| Mount Receiving Area | 100 |
| Single Wrap Removal Band | 110 |
| 1^{st} end | 111 |
| Elongated section | 112 |
| 2^{nd} end | 113 |
| Attachment to 2^{nd} section | 114 |
| of Elastomeric Strap | |
| Attachment to 2^{nd} section | 115 |
| of Elastomeric Strap | |
| Spool Attachment | 116 |
| Crimp | 117 |
| Double Wrap Removal Band | 120 |
| 1^{st} end | 121 |
| Elongated Section | 122 |
| 2^{nd} end | 123 |
| Spool Attachment | 124 |
| Attachment to Elastomeric | 125 |
| Strap | |
| Prolapse Removal Band | 130 |
| 1^{st} end | 131 |
| 2^{nd} end | 132 |
| Attachment to tool | 133 |
| Attachment to Elastomeric | 134 |
| Strap | |

### DETAILED DESCRIPTION OF THE INVENTION

At the outset, it should be understood that this invention comprises a method for removing antlers from an animal as well as an apparatus particularly adapted to make the removal pain and chemical free. The description which follows describes a preferred embodiment of the invention, and various alternative embodiments. It should be readily apparent to those skilled in the art, however, that various other alternative embodiments may be accomplished without departing from the spirit or scope of the invention.

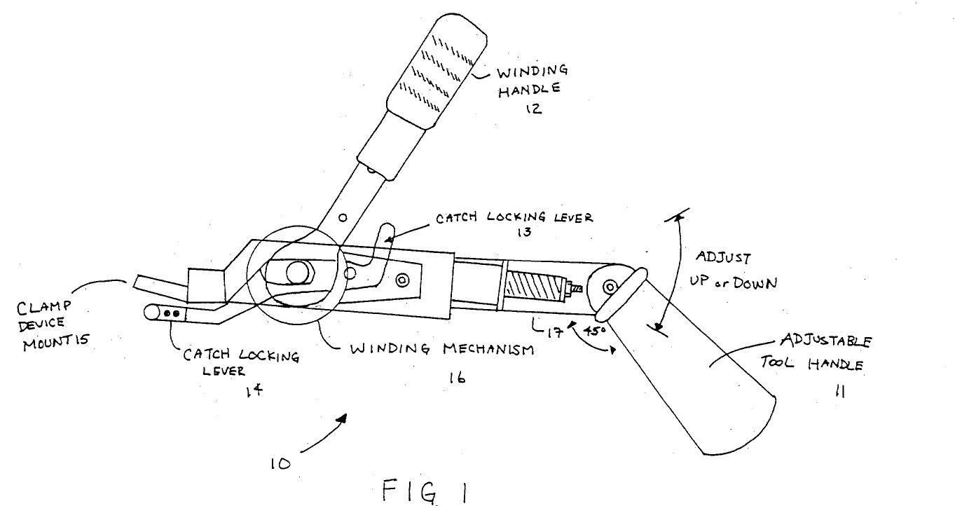

As shown in FIGS. 1 through 5, an apparatus constructed in accordance with an embodiment of the present invention is generally identified by the reference numeral 10. As shown, the tool 10 comprises a tool body 17, a winding mechanism 16, a clamp mount 15, and a catch locking lever 14. The tool body 17, which may comprise steel or other material having sufficient strength to withstand the forces encountered during the removal procedures, has a handle 11 at one end and a clamp mount 15 at the other end. In one embodiment, the tool handle 11 is pivotally mounted to the tool body 17 to allow the tool handle 11 to be adjusted up or down dependent on the approach of the user to the animal. This adjustability allows the user to approach the animal from a myriad of directions.

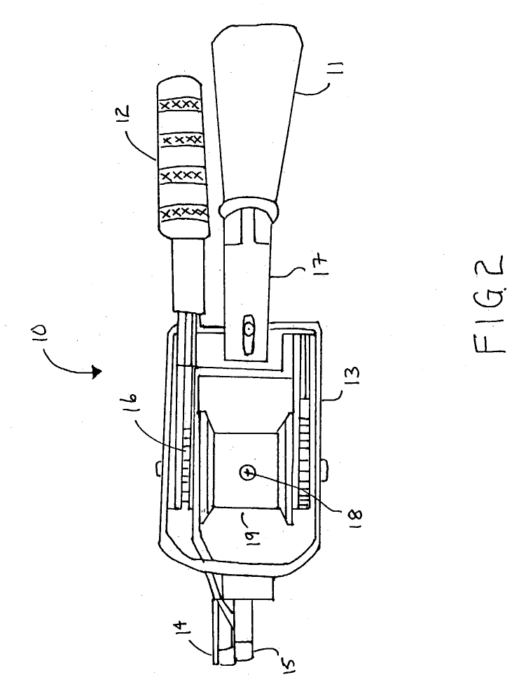

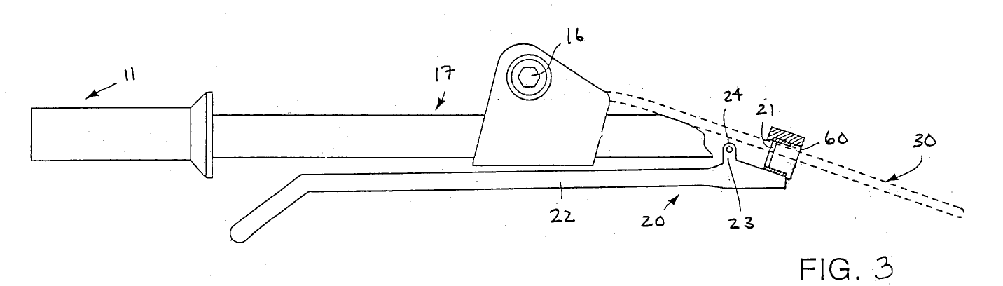

The winding mechanism can be hand operated or power-operated. An embodiment of the hand operated is shown in FIGS. 1 and 2. The winding mechanism, preferably, includes a ratchet mechanism to allow for one-way rotation of the winding mechanism and to allow for faster winding of the band about the spool. In a hand operated winding mechanism, the winding mechanism operatively engages a handle, which is rotatable in relation to the tool body. The winding mechanism 16 is attached to the tool body 17 by way of a frame which may comprise steel or other material of suitable strength. In one embodiment of the tool shown in FIGS. 1 and 2, the winding mechanism is along the same longitudinal axis as the tool body. In another embodiment of the tool, shown in FIGS. 3 through 5, the winding mechanism is mounted on a longitudinal axis parallel to that of the tool body.

In one embodiment of the present invention, the winding mechanism 16 comprises a spool 19 which is rotatably mounted in the frame. The spool also has an anchor 18, to which the removal band 110, 120 or 130 is attached. As can be readily appreciated, the rate at which the loop 30 is tightened will depend upon the diameter of the spool 19 and the speed of rotation of the spool 19. In addition, the tension which can be imparted to the strap 30 by winding the strap 30 a predetermined number of times around the spool 19 or, alternatively, by gauging the tension or torque exerted on the winding mechanism so that at a at a predetermined desired tension, the spool 19 is no longer rotated. The diameter of the spool 19 can therefore be selected to allow the desired rate and degree of tightening. Although it is believed that broad range of spool 19 diameters would provide adequate results, the illustrate spool 19 has a diameter between about ½ inch and ¾ inch. Such a diameter allows for rapid tightening of the loop 30 and allows the loop 30 to be sufficiently tightened to substantially cut off blood flow and systemic support to the body part to be removed.

Once the elastomeric loop has been tightened about the antler or other body part, loop size can be maintained by securely interconnecting portions of the loop adjacent the loop. The portions may be interconnected by used an adhesive, stapling, pinning or heat sealing the loop; binding portions of the loop with wire, rope or the like; or any other suitable method for securing the loop. The tool of the present invention can use a clamp device such as the one shown in FIGS. 6 through 9. In one of the illustrated embodiments as shown in FIG. 3, a crimping device 20 is provided to crimp a grommet 21 after the loop 30 has been tightened, thereby securing the loop 20. The grommet 21 preferably comprises a cylindrical structure having an interior passageway sufficient to allow passage of the loop 30 therethrough and can be from aluminum or other deformable material. The grommet 21 is designed so as to securely fasten the strap material without significantly damaging the material. The grommet 21 can be a completely enclosing angular structure or may alternatively be configured with side portions bendable to secure each individual end of the strap material, thus independently fastening each end without being dependent upon the adjacency of the other end to achieve a secure loop.

The crimping device shown in FIG. 3 comprises a lever 22 which is pivotally mounted on the body 17 by way of an attachment or connector 23 having a lock 24. The user can move the lever 22 from a retracted position to an extended position by urging the rearward end of the level downwardly. In the extended position, the forward end of the lever deforms the grommet. The connection 23 to the body is preferably positioned towards the forward end of the lever 22 so that a relatively small downward force exerted on the rearward end of the lever 22 by the use results in a greater crimping force on the grommet.

A tool constructed according to the present invention can additionally include a cutting assembly for cutting the strap material to release the strap material from the animal. Any device capable of cutting the band may be employed in accordance with the present invention.

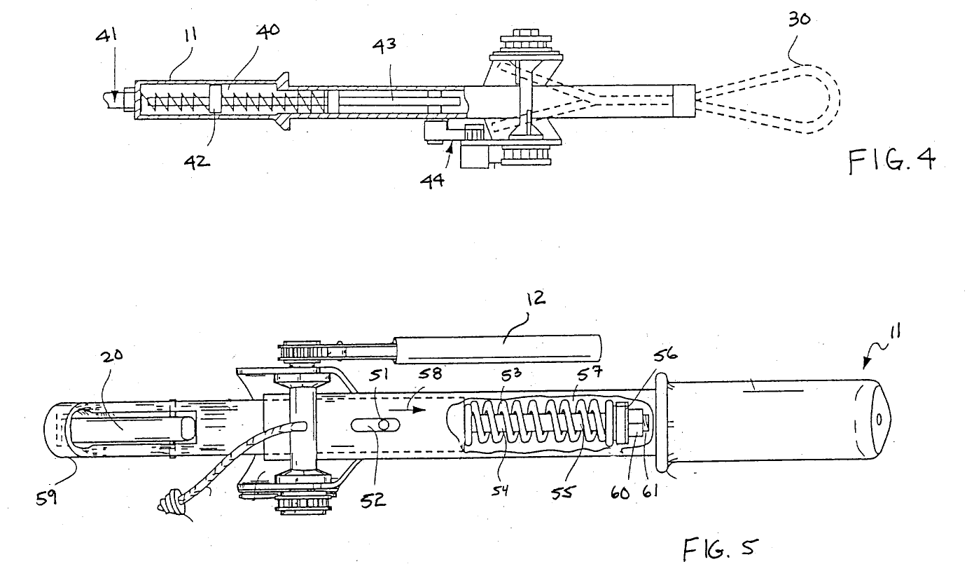

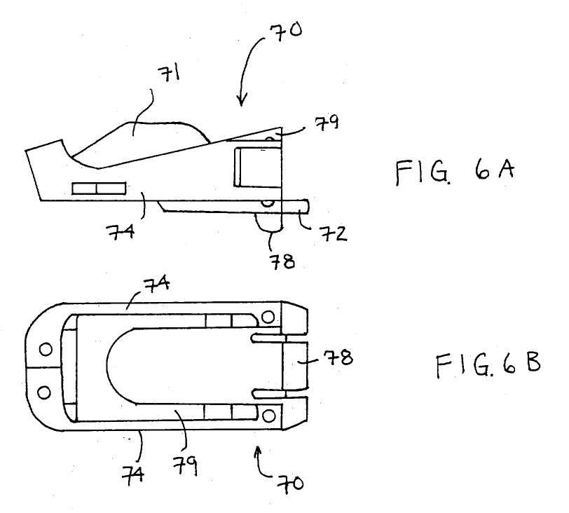

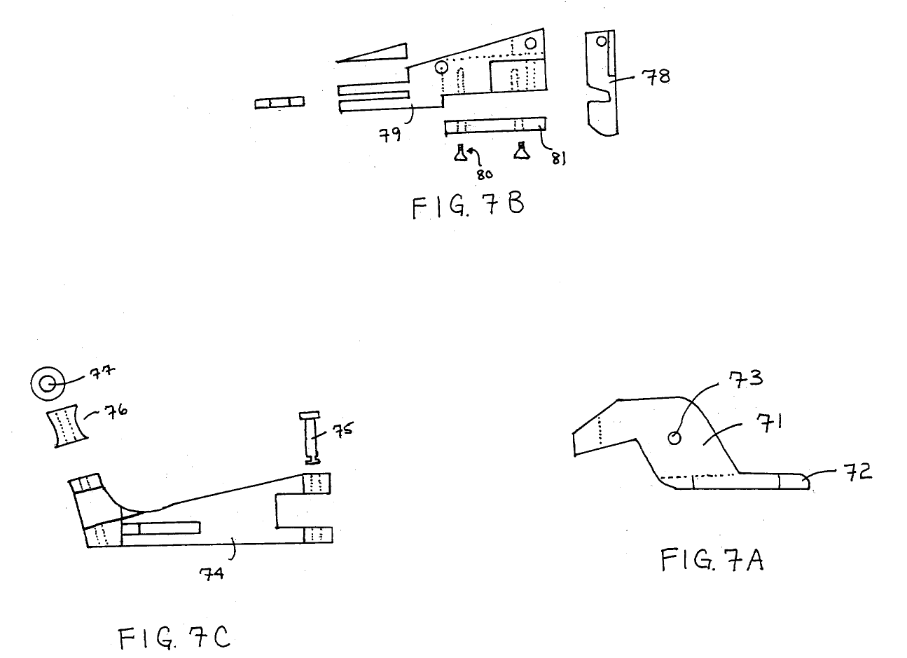

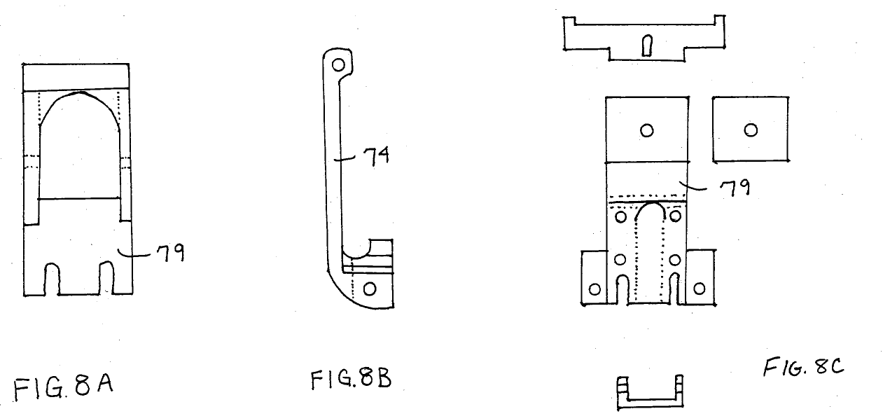

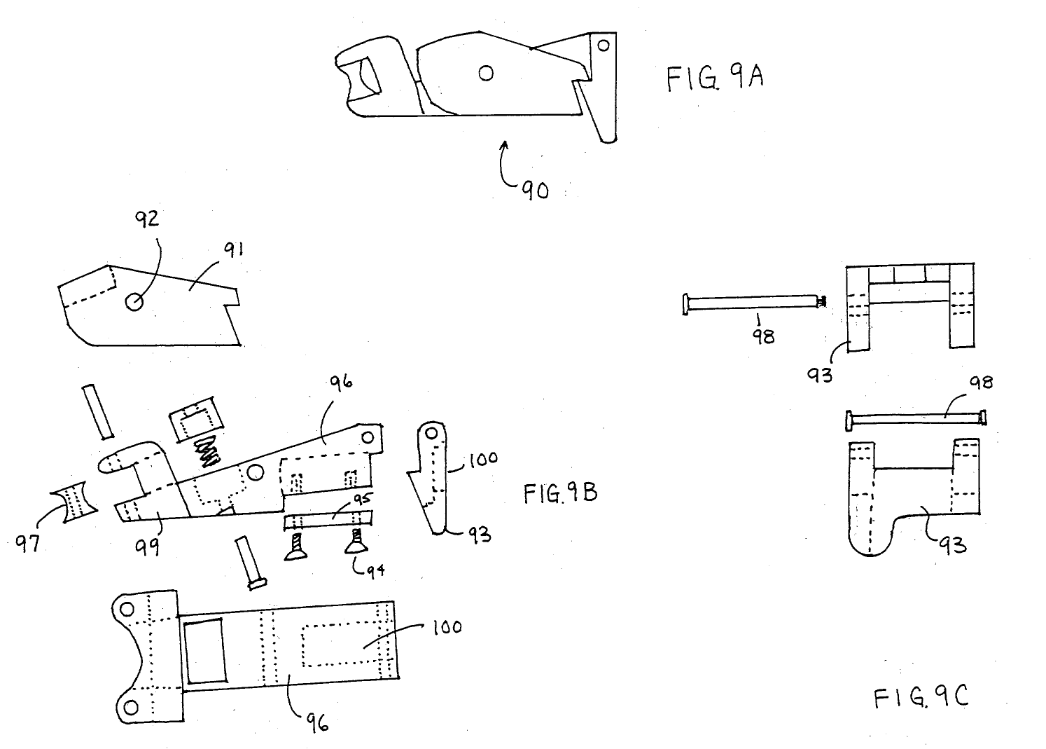

The winding mechanism 16 can also have a winding engagement site operatively connected both to the spool and to a motor, either pneumatic, hydraulic or electric. The motor can be integral to the tool or can be connected to the tool through a connection while the motor is in a remote location to the tool. The power tool or motor utilized must be capable of engaging the winding engagement site in a manner so that the winding mechanism is turned or rated, thus facilitating the accumulation of the removal band and the elastomeric strap material on the spool 19. An embodiment of the power-operated tool is shown in FIG. 4. In one embodiment, the winding mechanism comprises a suitably designed protuberance, such as a nut having several sides, or an indentation, similarly having a plurality of sides, that can operatively engage a suitably complementary power tool to achieve the rotation. The compatible multi-sided protuberance or indentation is configured such that the protuberance or indentation will match an engagement site on the power tool and the connection thereof will facilitate the powered rotation of the winding mechanism.

In another embodiment, the powered winding of the tool is achieved by the incorporation of a pneumatic or electrical device into the tool's 10 overall design so that a separate power tool need not be interconnected or engaged with a separate powered tool. As such, the pneumatic or electrical powered motor that is integral with the tool 10 is within the scope of the present invention. An illustrative embodiment is shown in FIG. 4.

With reference to FIG. 4, a pneumatic device 40 is incorporated into the handle 11 or the tool body 17 of the tool 10. An air supply 41 can be interconnected with one end of the handle 11 to operate an air piston 42 residing in the handle 11 or tool body 17. An air control valve can be located on the tool to control the winding operation so as to achieve desired winding of the elastomeric strap material 30 about the spool 19. In one embodiment, a drive linkage 44 between the pneumatic device 40 and the winding mechanism 16 is provided to permit operation of a ratcheted lever having an axis of rotation coincident with the axis of rotation of the spool 19. The drive linkage 44 is operatively attached to piston rod 43 which is driven by the air piston 42. Upon operation of the air piston 42, the piston rod 43 is moved back and forth within the body 17 of the device. Through such movement the drive linkage 44 communicated with the ratcheted lever to rotate the spool 19 in a desired direction. Winding of the elastomeric strap material 30 around the spool 19 can thus be accomplished by regulating the number of times the air piston 42 is forced forward and backward, thereby ratcheting the strap material 30 around the spool 19 to achieve a desired tension of the elastomeric loop 30. In another embodiment, an electric or pneumatic motor is incorporated into the tool 10 and is interconnected to a worm gear or other type of gearing mechanism. The worm gear is then operatively positioned to another gear interconnected to the spool 19. As can be appreciated by one skilled in the art, numerous types of gearing configurations may be implemented to transfer power from the motor to the spool 19.

In some applications, it is important to regulate the tightness or tension of an elastomeric loop 30 to prevent breakage thereof and prevent injury to the animal. The tightness of the strap material can be regulated by adjusting the amount of force communicated by the rotation of the winding mechanism 16 by a motor. This can be accomplished by, for example, a clutch mechanism either incorporated into the winding mechanism 16 or, alternatively, can be a feature of the power tool. In order to monitor the tightness or tension of the loop 30, a tension indicator device 50 can be provided. A shaft 55 can be provided within the body tool 17 that extends through at least a portion of the body tool 17. Inserted onto the shaft 55 is a compression spring 53 and a spring aligning spacer 54, wherein the spacer 54 is of sufficient length along the shaft 55 so that the central axis running the length of the compression spring 53 is substantially coincident with the length of the shaft 55. Additionally, also provided about the shaft 55 and adjacent to the compression spring 53 when the spring is being compressed as described hereinbelow. On the opposite side from the spring 53, washers 60 abut against a retaining member 56 that is fixed to the interior of the bore 57 of the body 17 (in which the tension indicator device is located), and that has a central opening of sufficient diameter to allow the threaded free end 61 to pass therethrough and yet effectively retain the washers 60 on the side of the retaining member 56 opposite the handle 11.

Additionally, on the side of the retaining member 56 having the handle 11, there is a bolt and washer assembly 60 secured to the threaded free end 61 of the shaft 55. Accordingly, given that the compression spring 53, when not under compression, is slightly longer than the distance between the end of body 17 and the washers 60, the assembled configuration as shown in FIG. 5 firmly secures the indicator device 50 into the body 17 in a manner that allows the device 50 to compress within the longitudinally extending bore 57 when an effective amount of force is provided on the body insert 304 in the direction of arrow 57. Attached to the device 50 toward the end is the tension indicator 51 In the present embodiment, the tension indicator 51 is viewable by a user of the apparatus 10 through a window or cut-out 52 in the body 17. Note that the tension indicator 51 can project outwardly from the device 50 and into the window or cut-out 52 so that this indicator remains aligned within the window or cut-out 52, and additionally assists in securing the device 50 within the body 17.

It will be appreciated that the removal band 110 and the elastomeric strap material 30 is pulled rearwardly through the clamp device towards the winding mechanism and around a spool of the winding mechanism in order to tighten the elastomeric strap material around the antler to be removed.

The clamp device of the present invention is shown in FIGS. 6 through 9, with FIG. 9 depicting a separate embodiment of the clamp device 70 or 90. The clamp device 70 or 90 is constructed of steel or other material having sufficient strength to withstand the forces encountered during the removal procedures. As discussed above, the clamp device 70 or 90 is mounted on the clamp mount 15 of the tool 10. The clamp mount 15 also operatively engages one end of the catch locking lever 14 of the tool 10 such that the catch locking lever prevents the clamping device from inadvertently moving or disengaging the tool. The release end 13 of the catch locking lever will allow the clamp device to be removed from the tool. The clamp device 70 or 90 has a receiving member adapted to fit over the clamp mount device 15 of the tool 10.

As shown in FIGS. 6 through 8, the clamping device 70 has a frame 79 that houses and anchors a pivot member 71. The pivot member is pivotally mounted to the frame through a fulcrum 73 to allow the pivot member to pivot about an axis. In the closed position of the pivot, the pivot member 71 will trap the removal band and/or strap material between the pivot member 71 and the frame 79. In the open position of the pivot, the pivot member 71 provides a passageway through which the removal band 110 or elastomeric strap 30 is passed before contact the spool 19 of the winding mechanism 16. The pivot member 71 also has a bar 72 that engages a lock 78 when the pivot member is in the closed position. The lock 78 is operatively engaged by the catch locking lever 14 of the tool 10. The lock 78, once engaged, prevents the clamping device from inadvertently moving to the open position.

The clamping device 70 additionally has arms 74 and rollers 77 that are moveable to many positions along a continuum between open and closed. The arms are secured to the frame through anchors 75, which can be screws or any other fixation means that will allow the arms to be movably secured to the frame. As the arms and rollers move to the closed position, the tightening of the strap 30 is facilitated and any gap between portions of the strap that form the loop around the antler is eliminated. The elimination of the gap between portions of the strap ensures the complete cut off of the blood supply to the antler, thus, additionally ensuring the pain free aspect of the method and apparatus described herein.

FIG. 9 shows another embodiment of the clamp device 90. The clamp device 90 also has a pivot member 91 with a fulcrum 92 for pivotally mounting the pivot member 91 to the frame 96. The clamp device 90 has rollers 97 which are used to assist the easy and smooth transition of the removal band and the portions of the elastomeric strap 30 through the band receiving area 99 in the frame 96. The pivot member 91 locks against the locking member 93, which additionally engages the catch locking lever 14 of the tool 10. The locking member 93 is pivotally mounted to the frame 96 at a fulcrum 98. The locking member 93 and the frame 96 have a mount receiving area 100 for mounting the clamp device to the tool 10 through the mount 15.

The elastomeric strap material, that forms a loop around the body part to be removed, can be made of any elastomeric material. Preferably, the elastomeric material has a hole in one end that will allow the removal band to be attached to the one end of the elastomeric strap material. However, such a hole is not necessary. The strap material can additionally have a small bore throughout the length of the material.

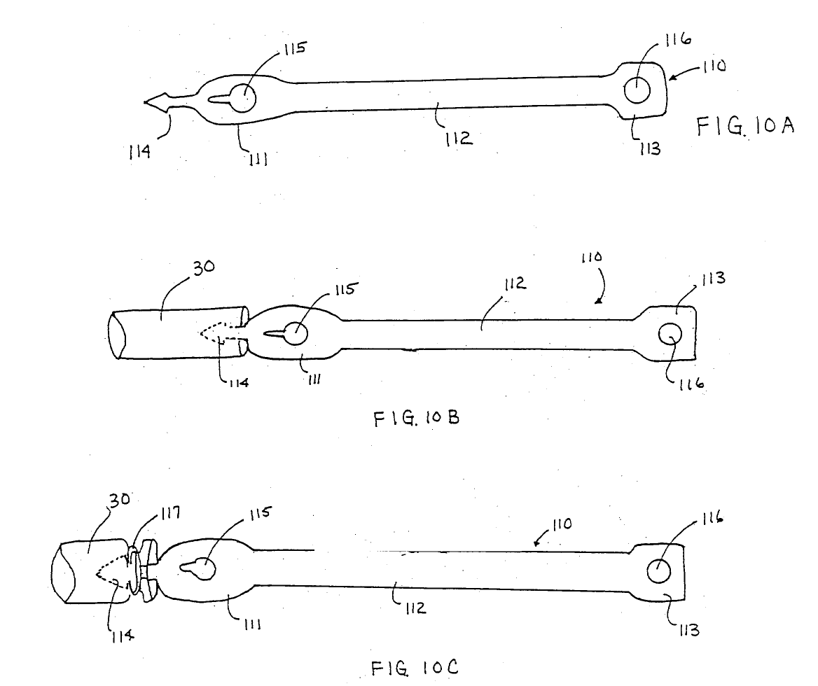

FIGS. 10A, 10B and 10C depict the removal band used in conjunction with the tool. FIG. 10A shows the removal band 110 which has an elongated section 112, a first end 111, a second end 113, and a attachment member 114 for one portion of the elastomeric strap. The removal band 110 can be made of any flexible, yet strong material, for example, plastic. The attachment member 114 is shown in FIGS. 10A through 10C as an arrow; however it will be obvious to one of skill in the art that any number of geometric shapes that will allow the elastomeric strap to be mounted on the removal band can be used. The removal band has an attachment to the 2^{nd} end of the elastomeric material. In the Figures, the attachment is shown as a hole through which the elastomeric strap is passed. The hole has a small slit attached to the hole. The elastomeric strap, once passed through the hole, is pushed or pulled into the small slit in order to keep the elastomeric strap from inadvertently coming loose or disengaging from the removal band. One of skill in the art will recognize that there are many different configurations for attaching the elastomeric strap material to the removal band. The removal band 110 further has an attachment member for the spool 124 that is, preferably, located opposite the attachment for the elastomeric strap material 114, 115. One of skill in the art will recognize that the spool attachment can be located anywhere on the removal band. The spool attachment 124 is shown, in the figures, as a hole which can be passed over and fits around the spool anchor 18.

FIG. 10B shows the elastomeric strap material attached to the removal band. Preferably, the elastomeric material is secured to the removal band by any securement means. FIG. 10C shows a wire band that encircles and crimps the elastomeric strap to the removal band. One of skill in the art will recognize that the wire band is only one of many different means to accomplish the securing of one end of the elastomeric material to the removal band.

FIG. 11 depicts another embodiment of the removal band 120 that will allow a double wrap of the elastomeric material around the body part to be removed, should such a double wrap be necessary. This embodiment of the removal band has an elongated section 122, a first end 121, a second end 123, a spool attachment 124, and two similar attachments for the ends of the elastomeric material 125. One of skill in the art will recognize that this removal band can also use different attachments such as those shown in FIGS. 10A through 10C.

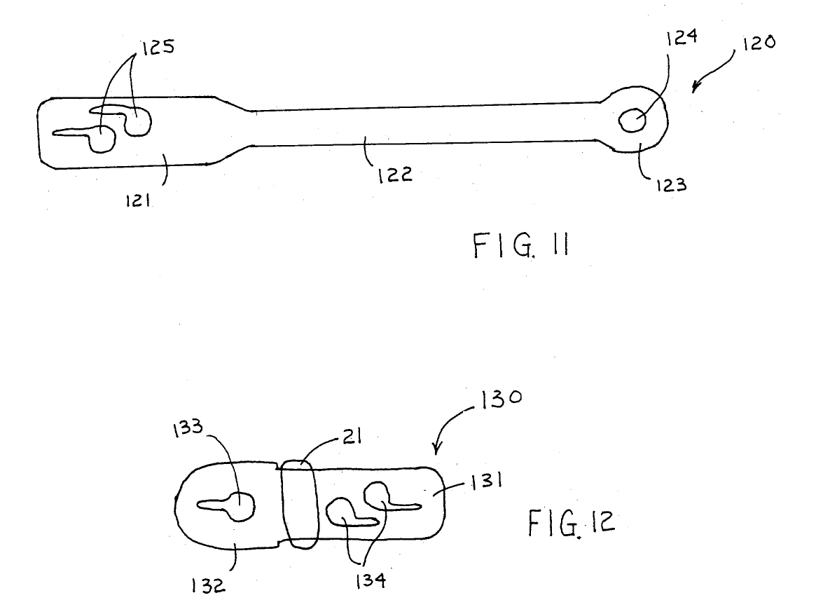

FIG. 12 depicts another embodiment of the removal band 130 that does not have the elongated section. This removal band is appropriate for use in removing prolapses. The removal band 130 has a first end 132, a second end 131, a spool attachment 133, and two similar attachments for the ends of the elastomeric material 134. The removal band shown in FIG. 12 also depicts the use of a grommet in connection with the band. One of skill in the art will recognize that a grommet can be used in connection with any of the embodiments of the removal band shown herein. One of skill in the art will also recognize that this removal band can also use different attachments such as those shown in FIGS. 10A through 10C.

For ease of discussion, the method of using the tool of the present invention will be discussed only in reference to the removal of an antler from an antlered animal. One of skill in the art will recognize that the method can be directly applied to other amputations and also the removal of prolapses in animals. In operation, a tool according to the present invention may be employed to assist in the removal of a antler as follows. Initially, a strap of elastomeric material is attached to a removal band. The strap material can be pre-attached to the removal band. The strap material is looped around the antler and secured to the removal band. A clamp device is mounted on the end of the tool and the removal band is passed through the clamp device and secured to the anchor on the spool of the winding mechanism. The winding mechanism is engaged and the removal band (and the loop) is wound around the spool. The winding mechanism contains to operate until loop is tightened around the antler enough to cut off the blood supply to the antler.

Once the loop is sufficiently tight, the clamp device is closed such that either the loop or the removal band are trapped between portions of the clamp device. The clamp device is removed from the tool and another clamp device and removal band are attached to the tool to begin the removal process of the other antler. Once the blood supply to both antlers has been cut off (usually occurring within four-five minutes of applying the straps to the antlers), the antlers are surgically removed with a saw or other appropriate tool.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

What is claimed is:

1. A method for removing a body part of an animal or a human, comprising:
attaching an elastomeric material to a removal tool;
forming a loop of the elastomeric strap material around the body part to be removed;
pulling said loop using said tool to tighten said loop around said body part;
clamping a device about said loop so that said loop maintains pressure about the body part until the body part can be removed through surgical means.

2. The method according to claim 1 further including attaching the elastomeric strap material to a removal band and attaching the removal band to a removal tool.

3. The method according to claim 1 wherein the pulling said loop includes winding said loop about a spool in the removal tool.

4. A removal tool, comprising:
a body having a length, said body having a means for receiving a removal band to which is attached to an elastomeric strap having a first and second portions;
a winding means operatively engaging said body for winding the removal band and the elastomeric strap material about said winding means and thereby increasing a tension on the strap for tightening about a body part of an animal.

5. The tool of claim 4, wherein said body includes a first and second body members that move together an increased tension is applied to the first portion of the elastomeric strap.

6. The tool of claim 5, wherein said body includes a compression spring for resisting a moving together of said body members.

7. The tool of claim 5, wherein said body members are in a telescoping relationship.

8. The tool of claim 5, further including a level for crimping a deformable grommet about said elastomeric strap material, said level adapted to engage the first body member.

9. The tool of claim 5, further including a cutting means for cutting said elastomeric strap material, wherein said cutting means is attached to said first body member.

10. The tool of claim 5 further including an indicating means for indicating when a desired tension of said elastomeric strap material has been achieved.

11. The tool of claim 4 further comprising a clamp device that is mounted to the tool, said clamp device securing the strap material once a sufficient tension has been achieved around a body part to be removed.

12. The tool of claim 11 further including a locking lever that removably secures the clamp device to the tool; said locking lever providing a release mechanism to allow the clamp device to be removed from the tool.

13. An removal band comprising a means for receiving a first and second portions of an elastomeric strap material and a means for engaging a winding means of an antler removal tool.

14. The removal band of claim 13 wherein the means for receiving also has a means for securing said elastomeric strap material to said band.

15. The removal band of claim 13 further comprising a second means for receiving a first and second portions of a second elastomeric strap material.

16. The removal band of claim 13 wherein the means for receiving the first portion of the elastomeric strap material is different from the means for receiving the second portion of the elastomeric strap material.

17. The removal band of claim 16 further including a means for securing the first portion of the elastomeric strap material to the removal band.

18. A method for removing an antler of an animal, comprising:
attaching an elastomeric material to a removal tool;
forming a loop of the elastomeric strap material around the antler to be removed;
pulling said loop using said tool to tighten said loop around said antler;
clamping a device about said loop so that said loop maintains pressure about the antler until the antler can be removed through surgical means.

19. The method according to claim 18 further including attaching the elastomeric strap material to a removal band and attaching the removal band to a removal tool.

20. The method according to claim 18 wherein the pulling said loop includes winding said loop about a spool in the removal tool.
